(19) United States
(12) Patent Application Publication
(10) Pub. No.: US 2020/0066374 A1
(43) Pub. Date: Feb. 27, 2020

(54) SYSTEMS AND METHODS EMPLOYING IMMORTALIZED INDUCED PLURIPOTENT STEM CELLS AS A PLATFORM FOR UNLIMITED LIFETIME GENETIC ANALYSIS

(71) Applicant: Orig3n, Inc., Boston, MA (US)

(72) Inventors: Robin Y. Smith, Boston, MA (US); Marcie A. Glicksman, Boston, MA (US); Kate Blanchard, Boston, MA (US)

(21) Appl. No.: 16/488,210

(22) PCT Filed: Feb. 23, 2018
(86) PCT No.: PCT/US18/19346

§ 371 Date: Aug. 22, 2019

Related U.S. Application Data
(60) Provisional application No. 62/537,266, filed on Jul. 26, 2017.
(60) Provisional application No. 62/463,481, filed on Feb. 24, 2017.

(51) Int. Cl. G16B 20/20; G16B 50/00; G01N 33/50; C12N 5/077; G16H 50/20; G16H 20/00

(52) U.S. Cl. CPC G16B 20/20; G16B 50/00; G16H 20/00; C12N 5/0657; G16H 50/20; G01N 33/5061

(57) ABSTRACT

Reserves of immortalized genetic material are stored in a bank for unlimited genetic analysis, tissue creation, lab-in-a-dish, and the like. The bank serves as a valuable platform for a variety of applications, including biological applications (e.g., genotyping, drug screening, personalized medicine), that require repeated testing, access to a biological sample, and maintenance over a long period of time. For example, a biological sample is stored as induced pluripotent stem cells (iPSCs) that can be used to generate an unlimited supply of genetic material when needed by a user. In this way, for instance, the generation of excess genetic material from additional samples provided by an individual can be avoided and costs to an organization associated with producing genetic material from biological sample can be deferred until necessary.

### CROSS REFERENCE TO RELATED APPLICATIONS

This application claims the benefit of U.S. Provisional Application No. 62/463,481 filed Feb. 24, 2017, and U.S. Provisional Application No. 62/537,266 filed Jul. 26, 2017, the contents of which are hereby incorporated by reference herein in their entirety.

### FIELD OF THE INVENTION

This invention relates generally to the use of immortalized induced pluripotent stem cells (iPSCs) and the genotyping data derived therefrom as a platform for unlimited lifetime genetic analysis, tissue creation, diagnosis and determination of treatment options of genetic diseases, lab-in-a-dish applications, and the like.

### BACKGROUND OF THE INVENTION

Genomes hold valuable information that can be used to better understand biological characteristics and traits of humans and animals. Much research is being conducted to establish relationships between the human genome and biological characteristics and traits, in particular. For example, many relationships between the variants of single nucleotide polymorphisms (SNPs) and their corresponding biological characteristics and traits have been established and many more possible relationships are currently undiscovered and under investigation.

Genetic diseases are hereditary diseases that result from an individual's particular genotype. Rare genetic diseases are a subset of genetic diseases with low prevalence that are often associated with complicated or fatal prognoses. Many individuals with a rare genetic disease die during childhood due to complications resulting from their disease. A large percentage of infant deaths in the United States are due to rare genetic diseases carried by infants. Some individuals do not exhibit signs or symptoms of their genetic disease for prolonged periods (e.g., into adulthood), at which point a genetic disease can have a significant impact on the individual's life.

Genetic screening can be performed to determine whether individuals have genetic diseases. Genotyping tests are performed using biological samples provided by individuals to determine whether the individuals have particular genetic diseases. However, genetic screening alone can be inconclusive, as is the case with many rare genetic diseases. For example, many rare genetic diseases have numerous subtypes based on different gene mutations that an individual may have, but frequently genotyping tests are unable to determine the particular mutation that an individual has. Furthermore, genetic screening cannot predict an individual's likelihood to respond to a particular treatment option. While genetic screening can be helpful in the advancement of personalized medicine, the science for the development of pharmaceuticals, biologics, and other treatments for genetic-related conditions is immature.

Determining a course of a treatment for an individual who has or may have a genetic disease or condition (e.g., a rare genetic disease), or a genetics-related or genetics-influenced disease or condition, typically requires a diminishing trial-and-error approach. In such approaches, a best guess is made followed by further refinements or substitutions based on an individual's response to each successive treatment. Functional assays may be used to determine treatment options by testing a large number of samples provided by an individual against a set of drugs. However, such functional assays are generic as they test the same sets of drugs for each individual.

Until recently, characterizing a genome was prohibitively expensive such that very few individual genomes had been fully or partially characterized. Techniques utilized in genotyping a genome required significant resources that limited genotyping to laboratory use in scientific research and related areas. Developments of cost-effective equipment and procedures for genotyping have made personal genotyping feasible. The output of genetic information from such genotyping procedures still requires expertise in the biological sciences to understand.

In order for individuals to gain an understanding of their genome, they may provide biological samples to an organization for genotyping. The individual can obtain their biological sample in any number of ways and send it to the organization. One or more assays are run to at least partially genotype the individual based on genetic material extracted from the biological sample. Typical genetic testing depletes the biological sample provided by an individual. In certain research or commercial settings, biological sample and/or genetic material is retained in order to allow for future genetic experiments or tests (e.g., genotyping) to be performed in the future. Each experiment or test uses some amount of genetic material, which slowly depletes the amount stored. Complete depletion of stored genetic material prevents further genetic testing from being performed, requiring an individual to provide an additional biological sample or additional genetic material to be derived from remaining stored biological sample.

There is a need for systems and methods that provide a long-term, unlimited supply of genetic material for genomic research, genetic testing, accurate diagnosis and treatment of genetic diseases, and personalized medicine development.

### SUMMARY

Reserves of immortalized genetic material are stored in a bank for unlimited genetic analysis, tissue creation, lab-in-a-dish, and the like. The bank serves as a valuable platform for a variety of applications, including biological applications (e.g., genotyping, drug screening, personalized medicine), that require repeated testing, access to a biological sample, and maintenance over a long period of time. For example, a biological sample is stored as induced pluripotent stem cells (iPSCs) that can be used to generate an unlimited supply of genetic material when needed by a user. In this way, for instance, the generation of excess genetic material from additional samples provided by an individual can be avoided and costs to an organization associated with producing genetic material from biological sample can be deferred until necessary.

Individuals may have biological sample material stored in cell repositories for purposes of supporting regenerative medicine. For example, induced pluripotent stem cells (iPS cells) may be produced from a blood sample (or other biological substance sample), then stored in a repository for ongoing and/or future use. By allowing a user (operator) to probe and/or access genetic material over a period of time (e.g., a lifetime), the user can appropriately plan and conduct experiments and/or tests on a stable, renewable material sample source without interruption to their workflow or delay in providing desired genetic (e.g., genomic) information to individuals (e.g., accurately diagnosing, e.g., monitoring, e.g., providing personalized treatment to individuals).

Immortalized cell lines, such as iPSCs, are populations of cells from a subject which would normally not proliferate indefinitely, but have been processed to evade normal cellular senescence and instead can keep undergoing division. The bank provides access to reserves of immortalized cells (e.g., iPSCs) for genetic analysis, and allows for a user to deliver information, without requesting any additional samples from the individual, over the lifetime of the individual, for example.

In recent years, there have been significant advances in the production of iPSCs from cells collected from a biological sample of a subject (e.g., blood cells). For example, iPSCs can be made by inserting copies of stem cell-associated genes—e.g., Oct 3/4, Sox 2, Klf4, and c-Myc (or Oct 3/4, Sox 2, Nanog, and Lin28)—into cells collected from the biological sample using viral vectors. See, for example, K. Okita, T. Ichisaka, and S. Yamanaka, “Generation of germline-competent induced pluripotent stem cells,” Nature, vol. 448, no. 7151, pp. 313-317, 2007; K. Okita, Y. Matsumura, Y. Sato et al., “A more efficient method to generate integration-free human iPS cells,” Nature Methods, vol. 8, no. 5, pp. 409-412, 2011.

iPS cells function like embryonic stem cells in that they can be differentiated into a variety of different cell types. For example, iPSCs can be differentiated to cardiomyocytes, neurons, keratinocytes (a cell type often affected in skin disorders), primordial germ cells, and other cell types. The differentiated cells may be used in ex vivo/in vitro disease modeling studies, for example, to determine response to various kinds of treatment (e.g., drugs or drug combinations). Such studies open up individualized medicine possibilities by permitting the ability to test the safety and/or effectiveness for many different treatment options, including non-intuitive drug combinations, for a given subject without risk to the subject. For example, iPSC-derived cardiomyocytes demonstrate synchronous contractions, and properties and/or functional behavior of the cells can be monitored under various conditions, e.g., exposure to various substances, e.g., drugs and drug combinations.

For example, certain embodiments described herein utilize iPSCs for screening an individual for a genetic condition, and/or for determining whether a particular treatment option will be safe and/or effective for the individual. One genetic condition for which these embodiments can be performed is Long QT. Long QT syndrome is a genetic cardiac condition that can cause episodes of erratic and/or fast heartbeat, which can trigger syncope or seizure. Long QT has the potential for causing sudden cardiac death. In the case of Long QT, exposure to certain substances/drugs can result in a dangerous, adverse reaction, so the ability to test drugs ex vivo is particularly important. Long QT is a condition with 12 subtypes based on 12 different gene mutations, but genetic screening results cannot be used to determine a subject's treatment options. A specific mutation cannot be identified in about 30% or more of subjects with clinically confirmed Long QT syndrome. Treatments range from low risk drug therapy to invasive heart surgery. Treatments that are safe and effective for certain individuals with Long QT may be dangerous for other individuals with Long QT. It is therefore important to assess treatment safety and efficacy for particular individuals.

Presented herein are ex vivo methods for screening and performing unlimited lifetime genetic analysis of an individual who has or may have a genetic disease or condition (e.g., a rare genetic disease). Individuals may be screened and analyzed for confirmation of the existence of the genetic disease or condition and/or for treatment options for the individual having the genetic disease or condition over long periods of time. In certain embodiments, the methods herein facilitate diagnoses and/or recommendations of treatment options made based on functional assays and genotyping data. Also presented herein are methods for creating a database of treatment options based on genotypes for use in the treatment of individuals with genetic diseases or conditions.

In one aspect, the invention is directed to an ex vivo method for detection of a genetic disease or condition in a subject (e.g., a rare genetic disease) and/or determination of one or more treatment options for the subject (e.g., screening for confirmation of the existence of the genetic disease or condition in the subject and/or screening for treatment options for the subject having the genetic disease or condition), the method comprising: accessing genotyping data for the subject (e.g., wherein the genotyping data is generated from measurements on genetic material derived from induced pluripotent stem cells) (e.g., wherein the measurements determine at least a portion of the subject's genotype corresponding to the genetic disease); obtaining a manufactured sample of induced pluripotent stem cells (iPSCs) derived from a biological sample provided by the subject; creating a plurality of testing samples, wherein each of the plurality of testing samples comprises ex vivo differentiated cells derived from the manufactured sample; performing a functional assay to test efficacies of one or more substances (e.g., drugs) (e.g., one or more individual substances or combinations of substances) using the plurality of testing samples (e.g., wherein each of the plurality of testing samples receives a distinct drug or a distinct combination of drugs e.g., a non-intuitive combination of drugs) (e.g., wherein the functional assay is performed automatically using a multi-well plate, e.g., a 96-well plate, e.g., using an automated testing platform)); and determining an existence or absence of the genetic disease or condition in the subject and/or determining one or more treatment options for the subject having the genetic disease or condition based at least in part on the functional assay and based at least in part, directly or indirectly, on the genotyping data for the subject (e.g., determining (e.g., automatically (e.g., by a processor of a computing device)) one or more recommended drugs and/or recommended combination of drugs for treatment of the genetic disease or condition based on the genotyping data and the functional assay).

In certain embodiments, the method comprises determining (e.g., selecting) the one or more substances for use in performing the functional assay (e.g., the one or more distinct drugs and/or distinct combinations of drugs for the plurality of testing samples) based at least in part on the genotyping data (e.g., wherein the genotyping data is derived from measurements of variants of the subject of one or more SNPs associated with the genetic disease).

In certain embodiments, the method comprises the step of: generating the sample of induced pluripotent stem cells from the biological sample provided by the subject.

In certain embodiments, the ex vivo differentiated cells are cardiomyocytes, wherein the ex vivo differentiated cells of each testing sample of the plurality of testing samples have a synchronous heartbeat.

In certain embodiments, the genetic disease or condition is Long QT syndrome.

In certain embodiments, the genetic disease or condition is a member selected from a group consisting of: Charcot-Marie-Tooth disorder, 47 XYY syndrome, Jacobs syndrome, Brugada syndrome, Turner syndrome, Fragile-X syndrome, Neurofibromatosis-1 muscular dystrophy (Duchenne and Becker type), Hereditary sensory and autonomic neuropathy 3, chromosome 22q11.2 deletion syndrome, alpha 1-antitrypsin deficiency, Long QT, and hereditary hemorrhagic telangiectasia.

In certain embodiments, the method comprises repeating the step of performing the functional assay over the lifetime of the subject.

In certain embodiments, the method comprises repeating the step of accessing the genotyping data over the lifetime of the subject.

In another aspect, the invention is directed to a method for creating a database of treatment options based on genotypes for use in treatment of subjects with genetic diseases or conditions over a lifetime of the subjects, the method comprising: accessing genotyping data for a plurality of subjects; obtaining, for each subject in the plurality of subjects, a manufactured sample of induced pluripotent stem cells (iPSCs) derived from a biological sample (e.g., a cheek swab, a blood sample, a urine sample, a tissue sample) provided by the subject; deriving ex vivo differentiated cells from each manufactured sample; performing a plurality of functional assays with the ex vivo differentiated cells and one or more substances (e.g., drugs) (e.g., testing one or more individual substances or combinations of substances); determining one or more treatment options (e.g., recommended drugs and/or combinations of recommended drugs) for each of a plurality of genotypes based on the plurality of functional assays; and generating a mapping (e.g., creating the database) by compiling determinations of the one or more treatment options and corresponding genotypes of the plurality of genotypes.

In certain embodiments, the method comprises performing (e.g., repeatedly), over the lifetime of each subject, the plurality of functional assays for each subject with the ex vivo differentiated cells of the subject, wherein performing the plurality of functional assays comprises: selecting the one or more substances for each subject based on the genotyping data for the subject.

In another aspect, the invention is directed to a method for creating a database of long-term treatment options based on genotypes for use in long-term treatment of subjects with genetic diseases or conditions over a long period of time (e.g., a lifetime of a plurality of subjects), the method comprising: accessing (e.g., repeatedly) genotyping data for a plurality of subjects over the long period of time (e.g., wherein the genotyping data is generated from measurements on genetic material derived from induced pluripotent stem cells) (e.g., wherein the measurements determine at least a portion of a genotype corresponding to a genetic disease or condition for each subject in the plurality of subject's); obtaining, for each subject in the plurality of subjects, a manufactured sample of induced pluripotent stem cells (iPSCs) derived from a biological sample provided by the subject; creating, for each subject in the plurality of subjects, a plurality of testing samples, wherein each of the plurality of testing samples comprises ex vivo differentiated cells derived from the manufactured sample for the subject; performing (e.g., repeatedly) a functional assay to test efficacies of one or more substances (e.g., drugs) (e.g., one or more individual substances or combinations of substances) for long-term treatment, over the long period of time, using the plurality of testing samples for each subject of the plurality of subjects, wherein each of the plurality of testing samples receives a distinct substance (e.g., a drug) or a distinct combination of substances (e.g., combination of drugs) (e.g., a non-intuitive combination of drugs), wherein the plurality of substances is selected based on the genotyping data for the subject (e.g., wherein the functional assay is performed automatically using a multi-well plate (e.g., a 96-well plate) (e.g., using an automated testing platform)); determining (e.g., automatically (e.g., by a processor of a computing device)) (e.g., repeatedly) one or more individual long-term treatment options (e.g., candidate drugs and/or combinations of candidate drugs) over the long period of time to recommend to treat the genetic disease or condition that each subject of the plurality of subjects has based, at least in part, on the functional assay for the subject; determining one or more preferred general long-term treatment options (e.g., recommended drugs and/or combinations of recommended drugs) for each of a plurality of genotypes based on the genotyping data for each subject of the plurality of subjects and the one or more individual long-term treatment options; and generating a mapping (e.g., creating the database) by compiling determinations of the one or more preferred general long-term treatment options and corresponding genotypes of the plurality of genotypes.

In certain embodiments, the, method comprises generating (e.g., repeatedly) the genotyping data for each subject of the plurality of subjects (e.g., over the lifetime of each subject) using the manufactured sample for each subject.

In certain embodiments, the method comprises determining (e.g., selecting) the one or more substances for use in performing the functional assay (e.g., the one or more distinct drugs and/or distinct combinations of drugs for the plurality of testing samples) for each subject in the plurality of subjects based at least in part on the genotyping data for the subject (e.g., wherein the genotyping data is derived from measurements of variants of one or more SNPs associated with the genetic disease or condition of the subject).

In certain embodiments, the method comprises monitoring the genotyping data of each subject in the plurality of subjects over the lifetime of each subject (e.g., repeatedly).

In another aspect, the invention is directed to a method of long term storage (e.g., automatically) (e.g., over a long period of time (e.g., a lifetime of an individual)) a plurality of reserves of immortalized cell lines (e.g., undifferentiated cells (e.g., induced pluripotent stem cells (iPSCs)) extracted and/or generated from biological samples of a plurality of individuals (e.g. saliva, e.g. blood, e.g. tissue, e.g. cheek cells (e.g. collected via a cheek (buccal) swab, e.g. urine, e.g. hair), the method comprising: storing, by a processor of a computer device, each of the plurality of reserves of immortalized cell lines corresponding to the plurality of individuals, said plurality of reserves contained in a bank (e.g., wherein the genotyping data is accessible (e.g., by a user) (e.g., repeatedly) over the long period of time).

In certain embodiments, the method comprises storing the plurality of reserves of immortalized cell lines in the bank.

In certain embodiments, the method comprises triggering (e.g., at one or more predetermined intervals of time, or upon occurrence of an event), by the processor, a notification (e.g., a graphical rendering in a software application) (e.g., that the reserve is to be re-queried for genetic analysis and/or screening) (e.g., that displays the genetic analysis and/or screening of each reserve) (e.g., the notification rendered for graphical presentation to a user, an operator, and/or the individual).

In certain embodiments, the triggering of the notification comprises issuing an alert [e.g. an email, e.g. a text message, e.g. an in-app notification, e.g. a push notification sent to a computing device (e.g. a smartphone, e.g. a tablet computer) of the individual associated with the reserve] [of availability of new genetic tests, advancements in treatments relevant to the particular individual (new drug available that is likely effective for the individual with particular genetics/particular haplotype, etc.)—access genetic material without requiring individual to provide another sample] [e.g., issuing an alert of low reserve amount (e.g. wherein the alert comprises an identification of the reserve and/or the individual associated with the reserve, e.g. wherein the alert comprises the meter value)] associated with a reserve of the plurality of reserves of immortalized cell lines.

In another aspect, the invention is directed to a repository of characterized immortalized cell lines (e.g., undifferentiated cells (e.g., induced pluripotent stem cells (iPSCs)) that are capable of being cultured (e.g., in vitro, in vivo), expanded (e.g., in vitro, in vivo), stored (e.g., frozen) (e.g., in a liquid nitrogen storage tank (e.g., at a temperature of about −195° C.), e.g., in a freezer (e.g., at a temperature from about −80° C. to about −20° C.)) (e.g., in a storage container having temperature and/or humidity control) (e.g., in a second storage tank in the case of a first storage tank temperature and/or humidity malfunction), differentiated (e.g., into tissue-specific cells (e.g., cardiomyocytes, hepatocytes) into blood cells, neurons), and queried over a long period of time (e.g., over a lifetime of an individual) for unlimited (e.g., repeated) genetic analysis, tissue creation, and/or lab-on-a-chip applications (e.g., wherein the repository is a biorepository for collecting, processing, storing, and/or distributing immortalized biospecimens, e.g., biological samples, iPSCs; e.g., wherein the repository is in electrical communication with one or more processors programmed for identifying, locating, and/or inventorying biospecimens in the repository, e.g., wherein the repository is outfitted with hardware, robotics, etc., for automated sample handling).

In another aspect, the invention is directed to a method for using a repository of characterized immortalized cell lines that are capable of being cultured, expanded, stored, differentiated, and queried over a long period of time for repeated genetic analysis, tissue creation, determination of treatment options, and/or lab-on-a-chip applications, the method comprising: for each of a plurality of individuals whose immortalized cell lines are contained in the repository, accessing, by a processor of a computer device, genotyping data corresponding to each of the immortalized cell lines stored in a reserve of the repository for use in in vitro and/or in vivo clinical procedures (e.g., gene therapy, cell or tissue transplant (e.g., mesenchymal stem cell transplant, bone marrow transplant, cosmetic surgery (e.g., cartilage grafts)).

In another aspect, the invention is directed to a method for using a repository of characterized immortalized cell lines that are capable of being cultured, expanded, stored, differentiated, and queried over a long period of time for repeated genetic analysis, tissue creation, determination of treatment options, and/or lab-on-a-chip applications, the method comprising: for each of a plurality of individuals whose immortalized cell lines are contained in the repository, accessing, by a processor of a computer device, genotyping data corresponding to each of the immortalized cell lines stored in a reserve of the repository for use in in vitro and/or in vivo pre-clinical studies (e.g., in vitro and in vivo screens, efficacy testing of medications, toxicity testing of medications, for use in personalized medicine, etc.).

In another aspect, the invention is directed to a system comprising a processor and a memory having instructions stored thereon, wherein the instructions, when executed by the processor, cause the processor to perform any of the methods described herein.

In another aspect, the invention is directed to a system for detection of a genetic disease or condition in a subject and/or determination of one or more treatment options for the subject, the system comprising: a processor; and a memory having instructions stored thereon, wherein the instructions, when executed by the processor, cause the processor to: access genotyping data for the subject; and determine an existence or absence of the genetic disease or condition in the subject and/or determine one or more treatment options for the subject having the genetic disease or condition, (i) based at least in part on a functional assay performed on a plurality of testing samples comprising ex vivo differentiated cells derived from a manufactured sample of induced pluripotent stem cells (iPSCs) derived from a biological sample provided by the subject, and (ii) based at least in part, directly or indirectly, on the genotyping data for the subject.

In certain embodiments, the instructions, when executed by the processor cause the processor to generate genotyping data from measurements of genetic material derived from the manufactured sample of iPSCs.

In certain embodiments, the functional assay is or has been performed to test efficacies of one or more individual substances or combination of substances using the plurality of testing samples, each of the plurality of testing samples receiving a distinct substance or a distinct combination of substances.

In certain embodiments, the instructions, when executed by the processor cause the processor to determine the one or more substances for use in performing the functional assay based at least in part on the genotyping data. In certain embodiments, the instructions, when executed by the processor cause the processor to generate genotyping data from measurements of variants of the subject of one or more SNPs associated with the genetic disease.

In certain embodiments, the ex vivo differentiated cells are cardiomyocytes, wherein the ex vivo differentiated cells of each testing sample of the plurality of testing samples have a synchronous heartbeat. In certain embodiments, the genetic disease or condition is Long QT syndrome.

In certain embodiments, the genetic disease or condition is a member selected from the group consisting of: Charcot-Marie-Tooth disorder, 47 XYY syndrome, Jacobs syndrome, Brugada syndrome, Turner syndrome, Fragile-X syndrome, Neurofibromatosis-1 muscular dystrophy (Duchenne and Becker type), Hereditary sensory and autonomic neuropathy 3, chromosome 22q11.2 deletion syndrome, alpha 1-antitrypsin deficiency, Long QT, and hereditary hemorrhagic telangiectasia.

In certain embodiments, the functional assay is or has been performed repeatedly. In certain embodiments, the instructions, when executed by the processor, cause the processor to repeatedly access the genotyping data for the subject.

In another aspect, the invention is directed to a system for creating a database of treatment options based on genotypes for use in treatment of subjects with genetic diseases or conditions, the system comprising: a processor; and a memory having instructions stored thereon, wherein the instructions, when executed by the processor, cause the processor to: access genotyping data for a plurality of subjects; determine one or more treatment options for each of a plurality of genotypes (i) based at least in part on a plurality of functional assays performed on ex vivo differentiated cells derived from a manufactured sample of induced pluripotent stem cells (iPSCs) derived from a biological sample provided by each subject in a plurality of subjects, and (ii) based at least in part, directly or indirectly, on the genotyping data for each of the plurality of subjects; and generate a mapping by compiling determinations of the one or more treatment options and corresponding genotypes of the plurality of genotypes.

In certain embodiments, the biological sample is a cheek swab, a blood sample, a urine sample, and/or a tissue sample. In certain embodiments, the plurality of functional assays are or have been performed to test efficacies of one or more individual substances or combination of substances using the differentiated cells, each of the plurality of functional assays receiving a distinct substance or a distinct combination of substances.

In certain embodiments, the instructions, when executed by the processor cause the processor to generate a database of mappings of determinations of the one or more treatment options and corresponding genotypes of the plurality of genotypes. In certain embodiments, the instructions, when executed by the processor cause the processor to: select the one or more substances for each subject in a plurality of subjects based on the genotyping data for the subject to perform, repeatedly, the plurality of functional assays for each subject with the ex vivo differentiated cells of the subject.

In another aspect, the invention is directed to a system for creating a database of long-term treatment options based on genotypes for use in long-term treatment of subjects with genetic diseases or conditions, the system comprising: a processor; and a memory having instructions stored thereon, wherein the instructions, when executed by the processor, cause the processor to: access genotyping data for a plurality of subjects; determine one or more individual long-term treatment options to recommend to treat the genetic disease or condition that each subject of the plurality of subjects has (i) based at least in part on a functional assay to test long-term treatment efficacies of one or more substances performed on a plurality of testing samples for each subject of the plurality of subjects, wherein the plurality of testing samples comprising ex vivo differentiated cells derived from a manufactured sample of induced pluripotent stem cells (iPSCs) derived from a biological sample provided by each subject in the plurality of subjects, and (ii) based at least in part, directly or indirectly, on the genotyping data for the subject; determine one or more preferred general long-term treatment options for each of a plurality of genotypes based on the genotyping data for each subject of the plurality of subjects and the one or more individual long-term treatment options; and generate a mapping by compiling determinations of the one or more preferred general long-term treatment options and corresponding genotypes of the plurality of genotypes.

In certain embodiments, the functional assay is or has been performed to test efficacies of one or more individual substances or combination of substances using the plurality of testing samples, each of the plurality of testing samples receiving a distinct substance or a distinct combination of substances.

In certain embodiments, the instructions, when executed by the processor cause the processor to generate a database of mappings of determinations of the one or more preferred general long-term treatment options and corresponding genotypes of the plurality of genotypes. In certain embodiments, the instructions, when executed by the processor cause the processor to generate genotyping data for each subject of the plurality of subjects (e.g., based on data (e.g., corresponding to measurements) obtained) using the manufactured sample for each subject. In certain embodiments, the instructions, when executed by the processor cause the processor to generate genotyping data for each subject in the plurality of subjects from measurements (e.g., data corresponding to measurements) of at least a portion of the subject's genetic material corresponding to a genetic disease or condition. In certain embodiments, the instructions, when executed by the processor cause the processor to generate genotyping data from measurements (e.g., data corresponding to measurements) of variants of one or more SNPs associated with the genetic disease or condition of the subject.

In certain embodiments, the instructions, when executed by the processor cause the processor to determine the one or more substances for use in the functional assay for each subject in the plurality of subjects based at least in part on the genotyping data for the subject. In certain embodiments, the instructions, when executed by the processor cause the processor to repeatedly monitor the genotyping data of each subject in the plurality of subjects.

In another aspect, the invention is directed to a system of long-term storage of a plurality of reserves of immortalized cell lines extracted and/or generated from biological samples of a plurality of individuals, the system comprising: a processor; and a memory having instructions stored thereon, wherein the instructions, when executed by the processor, cause the processor to store genotyping data corresponding to each of the plurality of reserves of immortalized cell lines contained in a bank corresponding to the plurality of individuals.

In certain embodiments, the system further comprises long-term storage of the plurality of reserves of immortalized cell lines in the bank. In certain embodiments, the plurality of reserves of immortalized cell lines comprise induced pluripotent stem cells (iPSCs) and/or undifferentiated cells. In certain embodiments, the plurality of reserves of immortalized cell lines comprise immortalized cell lines derived from saliva, blood, tissue, cheek cells, urine, and/or hair samples of the plurality of individuals.

In certain embodiments, the instructions, when executed by the processor, cause the processor to trigger a notification. In certain embodiments, the instructions, when executed by the processor cause the processor to trigger the notification at one or more predetermined intervals of time, or upon occurrence of an event. In certain embodiments, the notification comprises issuing an alert associated with a reserve of the plurality of reserves of immortalized cell lines. In certain embodiments, the instructions, when executed by the processor cause the processor to issue the alert, wherein the alert is an alert of low reserve amount, wherein the alert of low reserve amount comprises an identification of the reserve, an individual associated with the reserve, and/or a meter value associated with a low reserve amount associated with the individual. In certain embodiments, the instructions, when executed by the processor cause the processor to issue the alert, wherein the alert is an alert of new genetic tests, advancements in treatments relevant to an individual associated with the reserve, and/or access genetic material without requiring individual to provide another sample.

In another aspect, the invention is directed to a system for using a repository of characterized immortalized cell lines that are capable of being cultured, expanded, stored, differentiated, and queried over a long period of time for repeated genetic analysis, tissue creation, determination of treatment options, and/or lab-on-a-chip applications, the system comprising: a processor of a computing device; and a memory comprising instructions stored thereon, wherein the instructions, when executed by the processor, cause the processor to: access, for each of a plurality of individuals whose immortalized cell lines are contained in the repository, genotyping data corresponding to each of the immortalized cell lines stored in a reserve of the repository for use in in vitro and/or in vivo clinical procedures.

In certain embodiments, the clinical procedures comprise gene therapy, cell or tissue transplant, mesenchymal stem cell transplant, bone marrow transplant, and cosmetic surgery.

In another aspect, the invention is directed to a system for using a repository of characterized immortalized cell lines that are capable of being cultured, expanded, stored, differentiated, and queried over a long period of time for repeated genetic analysis, tissue creation, determination of treatment options, and/or lab-on-a-chip applications, the system comprising: a processor of a computing device; and a memory comprising instructions stored thereon, wherein the instructions, when executed by the processor, cause the processor to: access, for each of a plurality of individuals whose immortalized cell lines are contained in the repository, genotyping data corresponding to each of the immortalized cell lines stored in a reserve of the repository for use in in vitro and/or in vivo pre-clinical studies.

Elements of embodiments involving one aspect of the invention (e.g., methods) can be applied in embodiments involving other aspects of the invention (e.g., systems), and vice versa.

### DEFINITIONS

In order for the present disclosure to be more readily understood, certain terms used herein are defined below. Additional definitions for the following terms and other terms may be set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents.

Associate, Associated with: As used herein, the terms “associate,” and “associated with,” as in a first data structure is associated with a second data structure, refer to a computer representation of an association between two data structures or data elements that is stored electronically (e.g. in computer memory).

Biological material: As used herein, the term “biological material” refers to material extracted or derived from a biological sample that is used in a genotyping test or as a precursor material to a material used in a genotyping test. Biological material may be processed prior to being used to perform a genotyping test. In certain embodiments, biological material is DNA. In certain embodiments, biological material is RNA.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise saliva (e.g., collected via a cheek (buccal) swab), urine, hair, hair follicle cells, or blood. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate device and/or method. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as isolation and/or purification of certain components, etc.

Genotyping test: As used herein, the term “genotyping test” refers to a set of genotyping measurements used to determine information about an individual's genotype. A genotyping test is performed to measure one or more genes and/or SNPs.

Genotyping data: As used herein, the term “genotyping data” refers to data obtained from measurements of a genotype. In certain embodiments, genotyping data describes an individual's phenotype. Genotyping data may be measurements of particular genes (e.g., portions of an individual's genetic sequence, e.g., DNA sequence), SNPs, or variants of SNPs. In certain embodiments, genotyping data is obtained from a multi-gene panel. In certain embodiments, genotyping data is generated in response to a purchase or request by an individual. In certain embodiments, genotyping data comprises data for a portion of a genotype (e.g., of an individual). In certain embodiments, genotyping data comprises all available measurements of a genotype (e.g., of an individual).

“Organization”: As used herein, the term “organization” refers to an entity that performs genetic tests or otherwise uses or consumes stored biological samples and/or genetic material. The entity may be a company, individual, research group, research laboratory, non-profit organization, laboratory, hospital, medical organization, or medical testing facility. In certain embodiments, an organization performs genetic tests for research purposes. In certain embodiments, an organization performs genetic tests as a service or part of a service requested or purchased by an individual. In certain embodiments, the genetic tests an organization performs are genotyping tests.

“Bank”: As used herein, the term “bank” refers to a system, apparatus, or location where genetic material and/or biological sample is stored. Genetic material may be derived (e.g., extracted) from a biological sample provided by an individual to the organization that owns and/or operates the bank. In certain embodiments, biological samples are stored in a bank separate from a bank that stores genetic material extracted therefrom.

Graphical Control Element: As used herein, the term “graphical control element” refers to an element of a graphical user interface element (e.g., widget) that may be used to provide user and/or individual input. A graphical control element may be a textbox, dropdown list, radio button, data field, checkbox, button (e.g., selectable icon), list box, or slider.

“Individual”: As used herein, the term “individual” refers to someone who provides a biological sample to an organization for use in genetic testing and/or experimentation.

“User”: As used herein, the term “user” refers to someone associated with an organization who conducts and/or assists in conducting genetic testing and/or experimentation. In certain embodiments, a user is employed by an organization.

“Reserve”: As used herein, the term “reserve” refers to an amount of genetic material stored in a bank.

“Meter value”: As used herein, the term “meter value” refers to a value that reflects the amount of genetic material in a reserve stored in a bank. A meter value may be a percentage of a maximum capacity for a reserve, a value empirically measured or estimated from the amount of genetic material in a reserve, or a value that indicates fullness of the capacity of a reserve (e.g., a number on a scale from 1-5 or 1-10).

Product, Genetic Profile Product, Personal Genetic Profile Product: As used herein, the terms “product,” “genetic profile product,” and “personal genetic profile product,” refer to a data structure corresponding to (e.g. that is used to represent) a general class of health-related traits and/or characteristics. In certain embodiments a product is associated with one or more categories that correspond to health-related traits and characteristics related to the general class of health-related traits and characteristics to which the product corresponds.

Variant: As used herein, the term “variant” refers to a specific variation of a specific SNP occurring in the genome of an organism. In certain embodiments, a variant is a specific combination of a first allele of a first copy of an individual's genetic material (e.g., corresponding to an individual's paternal DNA) and a second allele of a second copy of an individual's genetic material (e.g., corresponding to an individual's maternal DNA), as occurs in diploid organisms (e.g., humans).

Subject: As used herein, the term “subject” refers to a human, other animal, or plant. In certain embodiments, subjects are humans and mammals (e.g., mice, rats, pigs, cats, dogs, horses, and primates). In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals are, for example, rodents (e.g., mice, rats, hamsters), rabbits, primates, or swine such as inbred pigs and the like.

Substance: As used herein, the term “substance” refers to medical material administered to an individual or cells derived from an individual to diagnose or treat a genetic disease (e.g., a rare genetic disease). A substance may be a probe (e.g., a chemical probe), a drug, formulation, medicament, supplement, vitamin, biologic, chemical, antibody, active agent, or combinations thereof. A substance may be a liquid, a solid, a pill, a topical formulation, an injectable, or a spray.

### DESCRIPTION OF THE DRAWINGS

The Drawings, which are comprised of at least the following Figures, is for illustration purposes only, not for limitation.

FIG. 1 shows an illustrative network environment 100 for use in the methods and systems described herein.

FIG. 2 shows an example of a computing device 200 and a mobile computing device 250 that can be used in the methods and systems described in this disclosure.

FIG. 3 is a block diagram showing an ex vivo method for repeated lifetime genetic analysis of a subject over who has or may have a genetic disease or condition, according to an illustrative embodiment of the invention.

FIG. 4 is a block diagram showing a method for creating a database of treatment options based on genotypes for use in treatment of subjects with genetic diseases or conditions, according to an illustrative embodiment of the invention.

FIG. 5 is a block diagram showing a method for creating a database of long-term treatment options based on genotypes for use in long-term treatment of subjects with genetic diseases or conditions, according to an illustrative embodiment of the invention.

FIG. 6 is a block diagram showing a method for long-term storage of a plurality of reserves of immortalized cell lines extracted and/or generated from biological samples of individuals, according to an illustrative embodiment of the invention.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DESCRIPTION OF THE INVENTION

Presented herein are systems and methods related for acquiring, storing, maintaining, accessing, and using, over a long period of time (e.g., a lifetime of an individual), immortalized cells extracted and/or generated from biological samples of individuals (e.g. saliva, e.g. blood, e.g. tissue, e.g. cheek cells (e.g. collected via a cheek (buccal) swab, e.g. urine, e.g. hair).

In certain embodiments, an individual provides a biological sample to an organization for use in characterizing genetic characteristics of genetic material contained in the biological sample, and/or for creating immortalized iPSCs for use in drug or treatment development, tissue generation, and/or other personalized medicine applications throughout the lifetime of the individual. One or more biological samples is obtained from the individual, processed, and provided to the bank. Biological samples may be, for example, saliva, blood, tissue, cheek cells, urine, hair, or induced pluripotent stem cells (iPSCs) generated from adult cells. Such biological samples may be taken by any commonly known method such as, for example, a cheek swab. Biological samples may be biological samples of the individual or they may belong to a person or animal related to the individual. In some embodiments, biological samples are from a non-human animal. For example, an individual may supply a biological sample of their pet in order to understand genomic information about the pet to assist in providing better care. The animal may be a pet or may be an animal cared for by an individual. For example, the individual may be a veterinarian or a caretaker at a zoo charged with caring for the animal. In some embodiments, an individual provides a biological sample of a ward to whom the individual is a guardian. For example, a parent may supply a biological sample to understand genomic information about his/her child in order to improve his/her childrearing.

Individuals may also have an immortalized biological sample material stored in cell repositories for purposes of supporting regenerative medicine and for generating an unlimited supply of samples for the individuals. For example, induced pluripotent stem cells (iPS cells) may be produced from a blood sample (or other biological substance sample), then stored in a repository for ongoing and/or future use.

Biological samples contain genetic material (e.g., DNA, RNA) that can be characterized by the organization. During processing and for any length of time after, genetic material derived from biological samples may be stored in a bank. In certain embodiments, an individual provides a biological sample to a company in order for the company to extract DNA from the biological sample for use in genotyping. In certain embodiments, the extracted DNA is stored in a bank when not in use.

An organization may store the genetic material of a large number of individuals for a prolonged period of time. A large bank of genetic material requires an accurate record of the amount of genetic material stored for reference in planning, conducting, and logging genetic tests, assays, or other related experiments. The accurate record may be a database or array stored electronically on a computer for reference using a computer (e.g., over a web interface or on a locally run piece of software).

A database or array will store a meter value that reflects the amount of genetic material in a bank for each individual for which genetic material has been extracted (i.e., from a biological sample). In this way, a user can view a database (e.g., visualized on a display) that indicates the meter value for each individual with genetic material in the bank in order to monitor the amount of genetic material of one or more individuals possessed by the organization.

Over the lifetime of an individual, an immortalized iPSCs can serve as a platform for unlimited genetic analysis. In certain embodiments, a user runs a PCR-based SNP genotyping assay (e.g., a TaqMan™ SNP genotyping assay). In certain embodiments, a genotyping assay is performed by a user as prompted by an individual's request for additional genomic information (e.g., when the individual purchases or is given access to additional products in a personal genetic profile assessment). Personal genetic profile assessments and systems and methods for viewing them are described in U.S. Patent Application No. 62/436,947, filed on Dec. 20, 2016, entitled “Systems and Methods for Creation of Personal Genetic Profile Products,”, and U.S. patent application Ser. No. 15/445,752, filed on Feb. 28, 2017, entitled “Systems And Methods For Creation Of Personal Genetic Profile Products”, the contents of which are hereby incorporated by reference herein in their entirety. Genetic material is deposited into a bank as an immortalized biological material, for example, when an individual provides an organization with one or more additional biological samples. As genetic material for each individual is stored as a separate reserve in a bank, the reserve of genetic material of an individual may be withdrawn from or deposited into as desired without impacting the reserves of other individuals also stored in the bank.

### Cells and Tissues

In certain embodiments, the systems and methods described herein use immortalized iPSCs for unlimited tissue and/or organ creation. For example, iPSCs can be differentiated into progenitor cells, mesoderm cells (e.g., cardiac cells, e.g., skeletal muscle cells, e.g., tubule cells, e.g., red blood cells, e.g., smooth muscle cells), endoderm cells (e.g., lung cells, e.g., thyroid cells, e.g., pancreatic cells), and/or ectoderm cells (e.g., skin cells, e.g., neuron cells, e.g., pigment cells) to create unlimited tissue resources for functional screening (e.g., for diagnosis and/or personalized medicine) as described herein.

Generation and Differentiation Protocols for Immortalized iPSCs

Induced pluripotent stem cell (iPSC) generation protocols are described, for example, at https://www.thermofisher.com/us/en/home/references/protocols/cell-culture/stem-cell-protocols/ipsc-protocols.html, the contents of which is hereby incorporated by reference in its entirety. Induced pluripotent stem cell (iPSC) generation and differentiation protocols are described, for example, at http://www.sigmaaldrich.com/life-science/stem-cell-biology/ipsc/ipsc-protocols.html, the contents of which is hereby incorporated by reference in its entirety. Differentiation of iPSCs can be found, for example, in “Induction of Pluripotent Stem Cells from Adult Human Fibroblasts by Defined Factors”; Takahashi K., Tanabe K., Ohnuki M., Narita M., Ichisaka T., Tomoda K., Yamanaka S.; Cell Vol. 131, 861-872, November 2007”, the contents of which is hereby incorporated by reference in its entirety.

Storage of Immortalized iPSCs

Repositories (e.g., cell repositories, e.g., nucleic acid repositories) for storing biological sample material (e.g., cells, e.g., nucleic acids) can include liquid nitrogen storage tanks and/or other freezer systems. Liquid nitrogen tanks provide temperature (e.g., about −195° C.) and/or humidity control, and can be used to store, for example, immortalized cell lines (e.g., immortalized iPSCs) over a long period of time. Alternatively, biological material (e.g., nucleic acids) can be stored in freezer systems at higher temperatures (e.g., from about −80° C. to about −20° C.). Additional equipment, backup systems, software/inventory control systems, sample location systems, automated sample retrieval, etc. can be used for storage and/or maintenance of the biological sample material stored in the repositories. The described setup allows for backup systems (e.g., additional repositories) to be used if a given tank and/or freezer temperature control system and/or humidity control system malfunctions.

Moreover, the provided systems and methods can record and track, via a graphical user interface, biological samples (and biological material extracted therefrom) used to generate genotyping data, for example, as described in U.S. Application No. 62/485,778, entitled “CHAIN OF CUSTODY FOR BIOLOGICAL SAMPLES AND BIOLOGICAL MATERIAL USED IN GENOTYPING TESTS” and filed on Apr. 14, 2017, U.S. application Ser. No. 15/846,659 entitled “CHAIN OF CUSTODY FOR BIOLOGICAL SAMPLES AND BIOLOGICAL MATERIAL USED IN GENOTYPING TESTS” filed on Dec. 19, 2017, and International Application No. PCT/US17/67272 entitled “CHAIN OF CUSTODY FOR BIOLOGICAL SAMPLES AND BIOLOGICAL MATERIAL USED IN GENOTYPING TESTS” filed on Dec. 19, 2017, the contents of which are hereby incorporated by reference in their entirety.

For example, as biological samples are processed in several stages to extract biological material and perform genotyping tests, IDs are assigned to biological sample material for individuals as well as well plates used during processing of the biological sample material in order to organize the samples and the tests. Biological sample materials are assigned to well plates for use in extracting biological material. Biological sample material is assigned to genotyping plates for use in performing genotyping tests. By associating IDs corresponding to biological sample material with IDs for well plates or genotyping plates, respectively, a user can track which extractions and/or tests need to be performed as well as record which biological samples have been received or genotyping plates analyzed via a graphical user interface.

### Applications

### Functional Screening

In certain embodiments, a bank comprising immortalized iPSCs are used in ex vivo methods for screening an individual who has or may have a genetic disease or condition (e.g., a rare genetic disease) and/or for identifying treatment options for the individual, e.g., by performing tests using the iPSCs derived from the biological sample of the individual and/or any iPSC-derived cells. For example, Table 1 shows a non-exhaustive list of rare genetic diseases that may be screened for repeatedly (e.g., multiple times over a long period of time (e.g., over the lifetime of a subject with the genetic disease and/or condition)) to make a diagnosis and/or recommend a treatment utilizing certain embodiments described. Additionally, Table 2 shows a non-exhaustive list of common genetic diseases that may be screened for repeatedly (e.g., multiple times over a long period of time (e.g., over the lifetime of a subject with the genetic disease and/or condition)) to make a diagnosis and/or recommend a treatment utilizing an illustrative embodiment of the invention. Methods for determining treatment options for genetic diseases or conditions based on genotyping data are described in U.S. Patent Application No. 62/463,481, filed on Feb. 24, 2017, entitled “Methods for determining treatment options for genetic diseases or conditions based on genotyping data,” the contents of which are hereby incorporated by reference herein in their entirety.

Table 1 shows a list of rare genetic diseases, their prevalence in the U.S. and the area of the body the disease impacts

| | US | |
|---|---|---|
| Rare Genetic Disease | Prevalence | Area of the body impacted |
| Charcot-Marie-Tooth | 150,000 | neurological disease |
| Disorder | | |
| 47 XYY syndrome | 136,000 | extra copy of the Y chromosome in |
| | | each of a male's cells |
| Jacobs syndrome | 136,000 | Jacob's syndrome is a rare |
| | | chromosomal disorder that affects |
| | | males. It is caused by the presence |
| | | of an extra Y chromosome. |
| Brugada Syndrome | 136,000 | heart rhythm disorder that is |
| | | sometimes inherited |
| Turner Syndrome | 108,799 | chromosomal condition in females |
| Fragile-X Syndrome | 90,666 | inherited form of intellectual |
| | | disability |
| Neurofibromatosis-1 | 90,666 | inherited neurological disorder |
| Muscular dystrophy, | 77,714 | muscles - heart & lungs potential |
| Duchenne and Becker | | |
| type | | |
| Hereditary | 73,513 | sensory dysfunction (depressed |
| sensory and | | reflexes, altered pain and |
| autonomic | | temperature perception) and |
| neuropathy 3 | | varying degrees of autonomic |
| | | dysfunction (gastroesophageal |
| | | reflux, postural hypotension, |
| | | excessive sweating) |
| Chromosome 22q11.2 | 68,000 | deletion of a small piece of |
| deletion syndrome | | chromosome 22 - can affect any |
| | | area of the body |
| Alpha 1-Antitrypsin | 54,399 | lung & liver |
| Deficiency/Familial | | |
| emphysema | | |
| Marfan syndrome | 54,399 | affects the body's connective tissue |
| Hereditary | 54,399 | results in the development of |
| hemorrhagic | | multiple abnormalities in the blood |
| telangiectasia | | vessels |

Table 2 shows a list of rare genetic diseases, their prevalence in the U.S. and the area of the body the disease impacts

| Disease | US prevalence |
|---|---|
| Familial atrial fibrillation | 81,600,000 |
| Androgenetic alopecia | 35,000,000 |
| Otosclerosis | 27,200,000 |
| Amyotrophic lateral sclerosis, familial | 27,200,000 |
| Glucose-6-Phosphate Dehydrogenase | 27,200,000 |
| Deficiency | |
| Crouzon Syndrome | 15,999,999 |
| Red-green color blindness | 13,600,000 |
| Retinoblastoma | 8,160,000 |
| Amyotrophic lateral sclerosis type 1 | 8,160,000 |
| Parkinson's Disease | 6,300,000 |
| Hereditary nonpolyposis colon cancer | 5,440,000 |
| Von Willebrand disease | 2,999,999 |
| Deuteranopia | 2,720,000 |
| Protanopia | 2,720,000 |
| 2-methylbutyryl-coenzyme A | 1,088,000 |
| dehydrogenase deficiency$ | |
| Familial Mediterranean fever | 1,088,000 |
| Heritable Disorders of Connective Tissue | 999,999 |
| Hemochromatosis type 1 | 999,999 |
| Multiple endocrine neoplasia type 2 | 906,666 |
| Maple syrup urine disease | 706,493 |
| Polycystic kidney disease | 600,000 |
| Sickle Cell Anemia | 544,000 |
| Heterozygous Familial | 544,000 |
| Hypercholesterolemia | |
| Gaucher disease type 1 | 544,000 |
| Klinefelter syndrome | 544,000 |
| Autosomal dominant polycystic kidney | 539,999 |
| disease | |
| Triple-X syndrome | 272,000 |

Individuals may be screened over their lifetime for confirmation of the existence of the genetic disease or condition and/or for identification of treatment options (e.g., recently developed treatment options, new treatments, etc.) for the individual having the genetic disease or condition. In certain embodiments, the methods herein facilitate diagnoses and/or recommendations of treatment options be made based on functional assays and genotyping data. For example, many genetic diseases are based on multiple different gene mutations. Table 3 lists a summary of three rare genetic diseases and the number of gene mutations implicated in each disease. Recommendations of treatment options for these and other such multi-gene diseases may need to be made based on the specific combination of genetic mutations identified in an individual (e.g., the individual's genotyping data), and the response of the identified genotype (e.g., cells from the patient (e.g., iPSCs, iPSC-derived cells)) to various treatments and/or treatment combinations (e.g., functional assays). Further, these functional assays and genotyping data can be repeated over the lifetime of the individual as needed, without having to retrieve additional samples for the individual.

Table 3 shows a summary of three rare genetic diseases and the number of gene mutations an individual can have that can influence the manifestation of the disease

| | | Number of |
|---|---|---|
| | | gene mutations |
| Condition | Description | implicated |
| Long QT | Delayed repolarization of the heart | 12 |
| Syndrome | following a heartbeat leading to prolonged | |
| (LQTS) | QT intervals on ECG's associated with a | |
| | risk for serious ventricular arrhythmias | |
| Arrhythmo- | A progressive disorder characterized by | 8 |
| genic Right | fibrofatty replacement of the myocardium, | |
| Ventricular | predisposing to ventricular tachycardia and | |
| Dysplasia | sudden death in young individuals and | |
| (ARVD) | athletes | |
| Familial | Associated with reduced left ventricular | At least 50 |
| dilated | function or systolic function leading to | |
| cardiac | enlargement of the heart | |
| myopathy | | |
| (FDC) | | |

Of particular emphasis here is the combination of genotyping data screening (e.g., using a gene panel, multi-gene panel) with ex vivo tests conducted on differentiated cells derived from iPSCs. This combination of steps permits routine, widespread screening of subjects (e.g., large populations, e.g., at least 100 k subjects, at least 500 k subjects, at least 1M subjects, at least 10M subjects, at least 50M subjects, at least 100M subjects, at least 200M subjects, or more subjects) for the existence of (or susceptibility for) a genetic condition, e.g., a rare genetic condition (see Tables 1 and 3), coupled with ex vivo testing of differentiated cells derived from iPSCs for a subset of the screened subjects. In some embodiments, the same biological sample may be used for both the screening step as well as (at least some of) the ex vivo testing of differentiated iPSCs. In some embodiments, the series of steps/procedures for ex vivo testing of the differentiated cells derived from iPSCs for a given subject is informed by the genotyping data for that subject. This permits the ability to identify subjects who have or are at risk of having a particular genetic condition, and to test the safety and/or effectiveness of many different treatment options for the screened subject, including non-intuitive drug combinations, without risk to the subject.

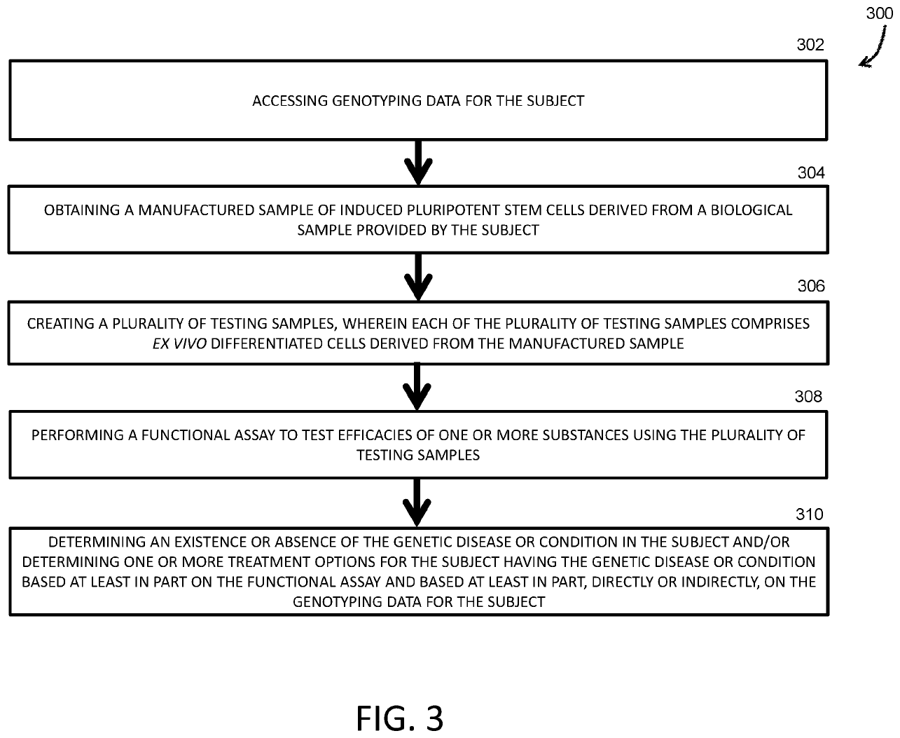

FIG. 3 is a block diagram showing a method 300 for detection of a genetic disease or condition in a subject and/or determination of one or more treatment options for the subject. In step 302, a processor of a computing device accesses genotyping data of the subject. In another step 304, a manufactured sample of induced pluripotent stem cells (iPSCs) derived from a biological sample provided by the subject is obtained. In order to test various potential treatments (e.g., for effectiveness and safety in treating the genetic disease or disorder), in step 306, a plurality of testing samples, each of the plurality of testing samples comprising ex vivo differentiated cells derived from the manufactured sample are created. In another step 308, a functional assay to test efficacies of one or more substances using the plurality of testing samples is performed. The existence or absence of the genetic disease or condition in the subject and/or one or more treatment options for the subject having the genetic disease or condition is then determined, in step 310. This determination is based at least in part on the functional assay, and based at least in part, directly or indirectly, on the genotyping data for the subject. The manufactured sample of iPSCs may also be used to generate the genotyping data of the subject.

In certain embodiments, the ex vivo testing of differentiated iPSCs is conducted to identify a subset of screened individuals who have a particular genetic condition but who are responsive to a particular known treatment, e.g., a simple, effective treatment (e.g., the use of beta blockers for treatment of Long QT syndrome). This may permit that subset of individuals to avoid a more lengthy, potentially stressful period of waiting for information about treatment options following a determination from the screening step that the individual has the screened-for genetic condition. For other individuals, further ex vivo testing may be needed to identify treatment options. For example, for subjects with Long QT syndrome for whom beta blockers are not effective (or which may, in fact, be contraindicated), other treatments, for example, non-intuitive, personalized drug combinations, may be tested.

For example, in certain embodiments iPSCs derived from a biological sample of a subject with Long QT syndrome may be differentiated into cardiac cells (e.g., iPSC-derived cardiomyocytes). These differentiated cells may then be subjected to further ex vivo treatments, for example, new drugs and/or drug combinations, to test potential efficacies in treating the genetic disorder of the subject. Treatments that are not effective and/or that are contraindicated due to the genotype of the subject may be identified through these ex vivo tests. For example, in certain embodiments, drugs and/or drug combinations that aggravate the disease phenotype may be recognized by marked arrhythmogenicity characterized by triggered arrhythmias (e.g., single or multiple premature beats), and/or action-potential duration (APD) studies.

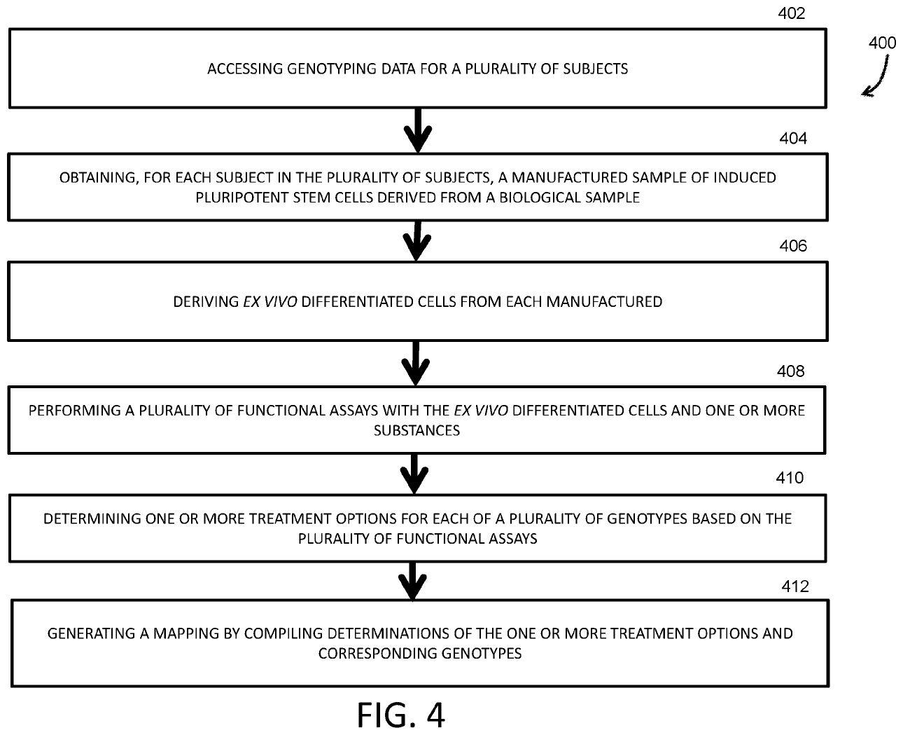

The systems and methods utilized for detection of a genetic disease or condition in a subject and/or determination of one or more treatment options for the subject may also be used for creating a database of treatment options based on genotypes. FIG. 4 is a block diagram showing a method 400 for creating a database of treatment options based on genotypes for use in treatment of subjects with genetic diseases or conditions (e.g., over a lifetime of the subjects), In one step 402, a processor of a computing device accesses genotyping data for the plurality of subjects. In another step 404, for each subject in the plurality of subjects, a manufactured sample of induced pluripotent stem cells (iPSCs) derived from a biological sample provided by the subject is obtained (e.g., generated, manufactured). These manufactured samples of iPSCs may then be used to derive, in step 406, ex vivo differentiated cells. In another step 408, a plurality of functional assays are performed with the ex vivo differentiated cells and one or more substances (e.g., individual and/or combinations of drugs and/or treatments). The different genotypes of the derived differentiated cells may respond differently to exposure the one or more substances. In another step 410, one or more treatment options (e.g., most effective and/or safe), for each of a plurality of genotypes based on the plurality of functional assays is determined. A processor of a computing device is used to generate a mapping of these determinations of the one or more treatment options and corresponding genotypes of the plurality of genotypes in step 412. Such mappings may be utilized to identify the most effective and/or safe treatment (e.g., individual treatment or combination of treatments) for a particular genetic disease or condition in a subject with a specific genotype.

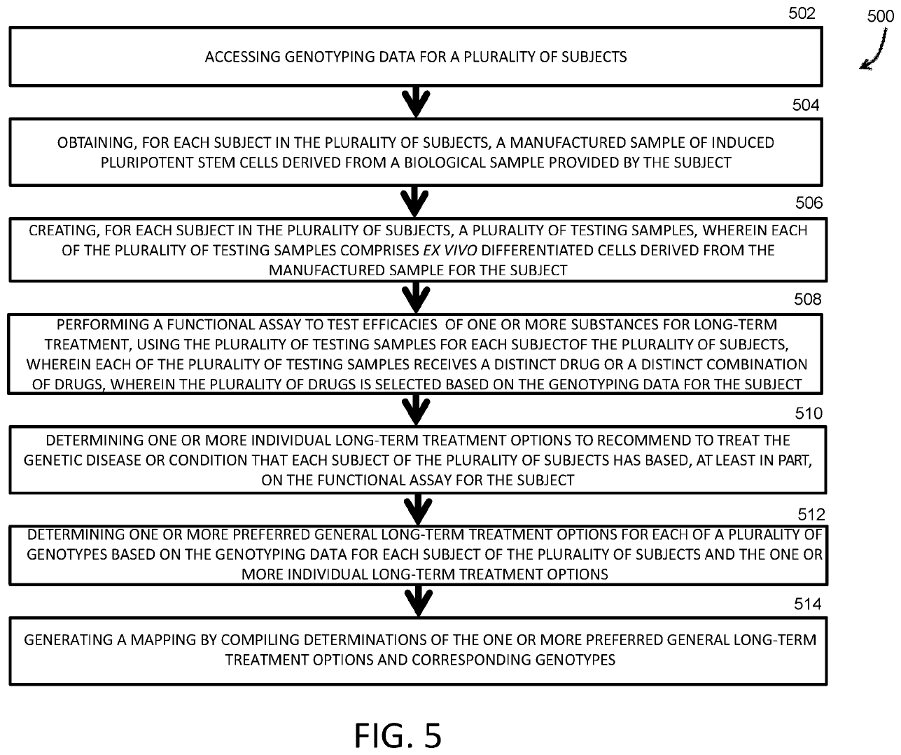

The systems and methods utilized for detection of a genetic disease or condition in a subject and/or determination of one or more treatment options for the subject may also be used for creating a database of long-term treatment options based on genotypes. FIG. 5 is a block diagram showing a method 500 for creating a database of long-term treatment options based on genotypes for use in long-term treatment of subjects with genetic diseases or conditions (e.g., chronic treatment, over a long period of time (e.g., over the lifetime of a subject)). In one step 502, a processor of a computing device accesses genotyping data for a plurality of subjects. In another step 504, for each subject in the plurality of subjects, a manufactured sample of induced pluripotent stem cells (iPSCs) derived from a biological sample provided by the subject is obtained. Then, for each subject in the plurality of subjects, a plurality of testing samples is created (506). Each of the plurality of testing samples comprises ex vivo differentiated cells derived from the manufactured sample for the subject. In another step 508, a functional assay to test efficacies of one or more substances for long-term treatment using the plurality of testing samples for each subject of the plurality of subjects is performed. Each of the plurality of testing samples receives a distinct drug or a distinct combination of drugs, and the plurality of drugs is selected based on the genotyping data for the subject. In another step 510, one or more individual long-term treatment options to recommend to treat the genetic disease or condition that each subject of the plurality of subjects has is determined, based at least in part, on the functional assay for the subject. Of all the various effective and/or safe long-term treatments, one or more preferred general long-term treatment options are determined for each of a plurality of genotypes based on the genotyping data for each subject of the plurality of subjects and the one or more individual long-term treatment options (512). A processor of a computing device is utilized to generate a mapping by compiling determinations of the one or more preferred general long-term treatment options and corresponding genotypes of the plurality of genotypes, in step 514. Such mappings may be utilized to identify the most effective and/or safe long-term treatment (e.g., individual treatment or combination of treatments) (e.g., over a long period of time (e.g., lifetime of a subject)) for a particular genetic disease or condition in a subject with a specific genotype.

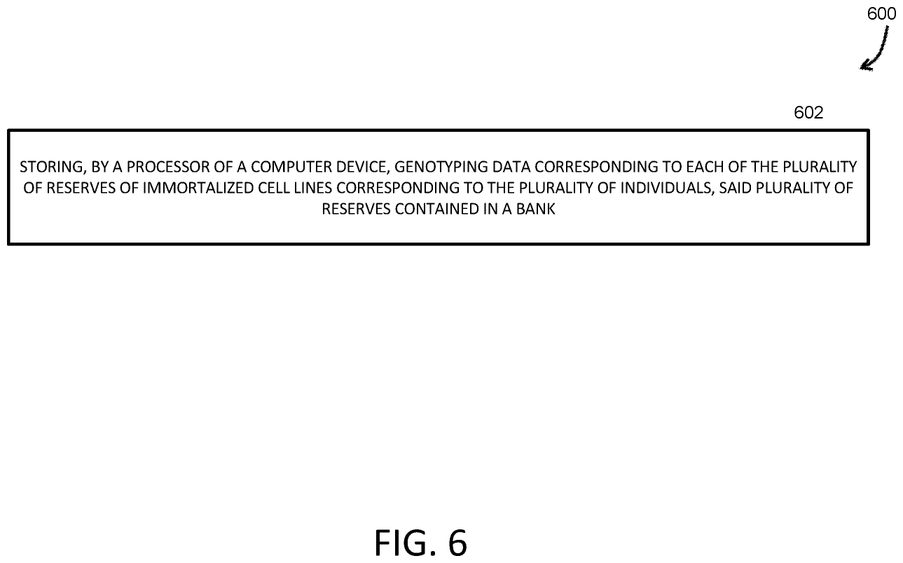

Turning to FIG. 6, which shows a block diagram of a method 600 for long-term storage of a plurality of reserves of immortalized cell lines extracted and/or generated from biological samples of individuals. In one step 602, using a processor of a computer device, to store genotyping data corresponding to each of the plurality of reserves of immortalized cell lines corresponding to the plurality of individuals, said plurality of reserves contained in a bank. The bank also stores the plurality of reserves of immortalized cell lines corresponding to the plurality of individuals.

### Isolating DNA Fragments in a Maternal Blood (Or Other Biological) Sample

In certain embodiments, a bank comprising immortalized iPSCs are used in systems and methods of isolating DNA fragments in a maternal blood (or other biological) sample, and performing genotyping of fetal DNA to screen for diseases and/or conditions. For example, an unborn child suspected of or susceptible to a genetic disorder can undergo personalized medicine and treatment prior to birth. Moreover, SNP-analysis of genotyping data acquired from a biological sample from the mother can provide recommendations of treatments to ensure that both mother and baby are not adversely affected by the treatment. Creation of tissues for baby prior to birth can also be generated based on genotyping data of fetal DNA.

Moreover, the fetal DNA can undergo SNP analysis, e.g., to determine types of foods and/or nutritious supplemental that the mother should eat for improved health of the mother and/or baby.

Use of iPSC Banks for Protection of Endangered Species and/or Zoological Preservation

In certain embodiments, a bank comprising immortalized iPSCs are used in methods for protecting endangered species and/or zoological preservation. iPSCs can be derived from animals, including, endangered species in an effort to preserve genetic materials from those animals (see, for example, Ben-Nun et al., “Induced pluripotent stem cells form highly endangered species”, Nature Methods, ACCEPTED 19 August; PUBLISHED ONLINE 4 Sep. 2011; DOI:10.1038/NMETH.1706). For example, upon notice of risk of a species becoming endangered, the bank can be used as a resource to provide genotyping data and/or a resource for tissue generation towards preserving the endangered species. Moreover, the provided systems and methods facilitate acquiring, storing, maintaining, accessing, sharing, and using, over a long period of time, iPSCs derived from endangered species.

### Illustrative Computer Network Environment

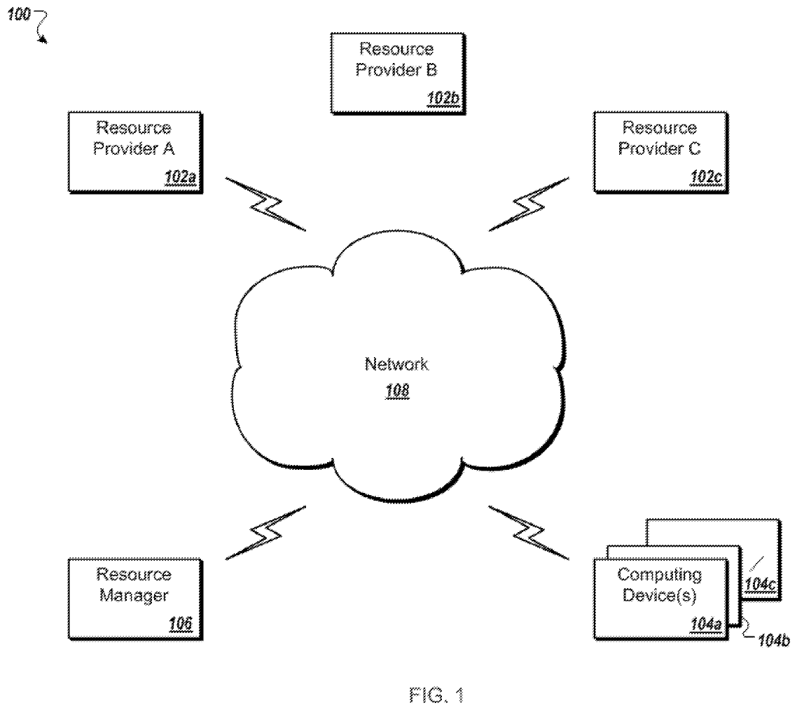

FIG. 1 shows an illustrative network environment 100 for use in the methods and systems described herein. In brief overview, referring now to FIG. 1, a block diagram of an exemplary cloud computing environment 100 is shown and described. The cloud computing environment 100 may include one or more resource providers 102a, 102b, 102c (collectively, 102). Each resource provider 102 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 102 may be connected to any other resource provider 102 in the cloud computing environment 100. In some implementations, the resource providers 102 may be connected over a computer network 108. Each resource provider 102 may be connected to one or more computing device 104a, 104b, 104c (collectively, 104), over the computer network 108.

The cloud computing environment 100 may include a resource manager 106. The resource manager 106 may be connected to the resource providers 102 and the computing devices 104 over the computer network 108. In some implementations, the resource manager 106 may facilitate the provision of computing resources by one or more resource providers 102 to one or more computing devices 104. The resource manager 106 may receive a request for a computing resource from a particular computing device 104. The resource manager 106 may identify one or more resource providers 102 capable of providing the computing resource requested by the computing device 104. The resource manager 106 may select a resource provider 102 to provide the computing resource. The resource manager 106 may facilitate a connection between the resource provider 102 and a particular computing device 104. In some implementations, the resource manager 106 may establish a connection between a particular resource provider 102 and a particular computing device 104. In some implementations, the resource manager 106 may redirect a particular computing device 104 to a particular resource provider 102 with the requested computing resource.

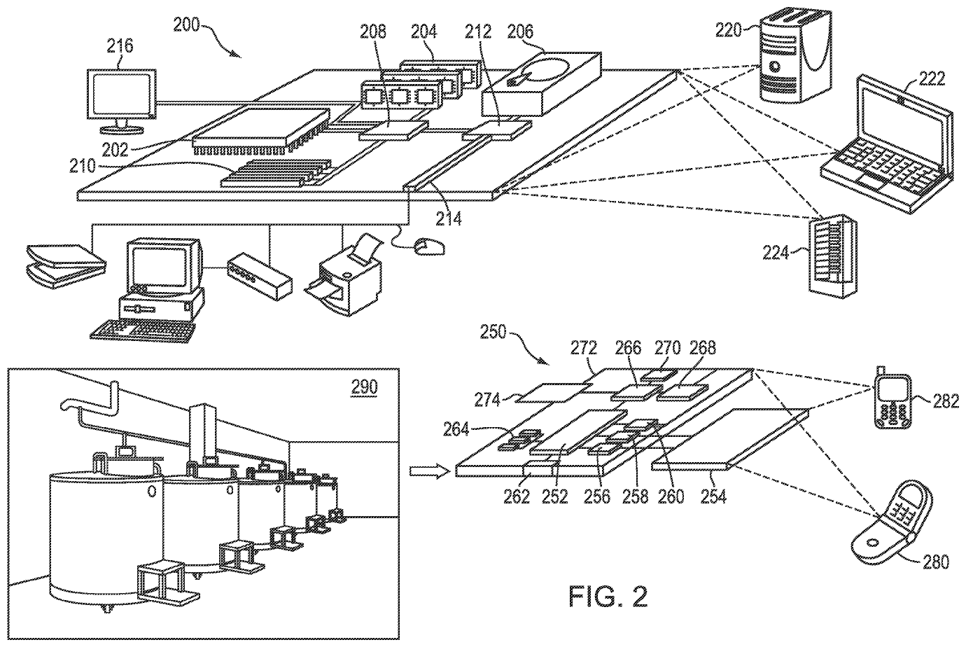

FIG. 2 shows an example of a computing device 200 and a mobile computing device 250 that can be used in the methods and systems described in this disclosure. The computing device 200 is intended to represent various forms of digital computers, such as laptops, desktops, workstations, personal digital assistants, servers, blade servers, mainframes, and other appropriate computers. The mobile computing device 250 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 200 includes a processor 202, a memory 204, a storage device 206, a high-speed interface 208 connecting to the memory 204 and multiple high-speed expansion ports 210, and a low-speed interface 212 connecting to a low-speed expansion port 214 and the storage device 206. Each of the processor 202, the memory 204, the storage device 206, the high-speed interface 208, the high-speed expansion ports 210, and the low-speed interface 212, are interconnected using various busses, and may be mounted on a common motherboard or in other manners as appropriate. The processor 202 can process instructions for execution within the computing device 200, including instructions stored in the memory 204 or on the storage device 206 to display graphical information for a GUI on an external input/output device, such as a display 216 coupled to the high-speed interface 208. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 204 stores information within the computing device 200. In some implementations, the memory 204 is a volatile memory unit or units. In some implementations, the memory 204 is a non-volatile memory unit or units. The memory 204 may also be another form of computer-readable medium, such as a magnetic or optical disk.

The storage device 206 is capable of providing mass storage for the computing device 200. In some implementations, the storage device 206 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 202), perform one or more methods, such as those described above. The instructions can also be stored by one or more storage devices such as computer- or machine-readable mediums (for example, the memory 204, the storage device 206, or memory on the processor 202).

The high-speed interface 208 manages bandwidth-intensive operations for the computing device 200, while the low-speed interface 212 manages lower bandwidth-intensive operations. Such allocation of functions is an example only. In some implementations, the high-speed interface 208 is coupled to the memory 204, the display 216 (e.g., through a graphics processor or accelerator), and to the high-speed expansion ports 210, which may accept various expansion cards (not shown). In the implementation, the low-speed interface 212 is coupled to the storage device 206 and the low-speed expansion port 214. The low-speed expansion port 214, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 200 may be implemented in a number of different forms, as shown in the figure. For example, it may be implemented as a standard server 220, or multiple times in a group of such servers. In addition, it may be implemented in a personal computer such as a laptop computer 222. It may also be implemented as part of a rack server system 224. Alternatively, components from the computing device 200 may be combined with other components in a mobile device (not shown), such as a mobile computing device 250. Each of such devices may contain one or more of the computing device 200 and the mobile computing device 250, and an entire system may be made up of multiple computing devices communicating with each other.

The mobile computing device 250 includes a processor 252, a memory 264, an input/output device such as a display 254, a communication interface 266, and a transceiver 268, among other components. The mobile computing device 250 may also be provided with a storage device, such as a micro-drive or other device, to provide additional storage. Each of the processor 252, the memory 264, the display 254, the communication interface 266, and the transceiver 268, are interconnected using various buses, and several of the components may be mounted on a common motherboard or in other manners as appropriate.

The processor 252 can execute instructions within the mobile computing device 250, including instructions stored in the memory 264. The processor 252 may be implemented as a chipset of chips that include separate and multiple analog and digital processors. The processor 252 may provide, for example, for coordination of the other components of the mobile computing device 250, such as control of user interfaces, applications run by the mobile computing device 250, and wireless communication by the mobile computing device 250.

The processor 252 may communicate with a user through a control interface 258 and a display interface 256 coupled to the display 254. The display 254 may be, for example, a TFT (Thin-Film-Transistor Liquid Crystal Display) display or an OLED (Organic Light Emitting Diode) display, or other appropriate display technology. The display interface 256 may comprise appropriate circuitry for driving the display 254 to present graphical and other information to a user. The control interface 258 may receive commands from a user and convert them for submission to the processor 252. In addition, an external interface 262 may provide communication with the processor 252, so as to enable near area communication of the mobile computing device 250 with other devices. The external interface 262 may provide, for example, for wired communication in some implementations, or for wireless communication in other implementations, and multiple interfaces may also be used.

The memory 264 stores information within the mobile computing device 250. The memory 264 can be implemented as one or more of a computer-readable medium or media, a volatile memory unit or units, or a non-volatile memory unit or units. An expansion memory 274 may also be provided and connected to the mobile computing device 250 through an expansion interface 272, which may include, for example, a SIMM (Single In Line Memory Module) card interface. The expansion memory 274 may provide extra storage space for the mobile computing device 250, or may also store applications or other information for the mobile computing device 250. Specifically, the expansion memory 274 may include instructions to carry out or supplement the processes described above, and may include secure information also. Thus, for example, the expansion memory 274 may be provided as a security module for the mobile computing device 250, and may be programmed with instructions that permit secure use of the mobile computing device 250. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 252), perform one or more methods, such as those described above. The instructions can also be stored by one or more storage devices, such as one or more computer- or machine-readable mediums (for example, the memory 264, the expansion memory 274, or memory on the processor 252). In some implementations, the instructions can be received in a propagated signal, for example, over the transceiver 268 or the external interface 262.

The mobile computing device 250 may communicate wirelessly through the communication interface 266, which may include digital signal processing circuitry where necessary. The communication interface 266 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 268 using a radio-frequency. In addition, short-range communication may occur, such as using a Bluetooth®, Wi-Fi™, or other such transceiver (not shown). In addition, a GPS (Global Positioning System) receiver module 270 may provide additional navigation- and location-related wireless data to the mobile computing device 250, which may be used as appropriate by applications running on the mobile computing device 250.

The mobile computing device 250 may also communicate audibly using an audio codec 260, which may receive spoken information from a user and convert it to usable digital information. The audio codec 260 may likewise generate audible sound for a user, such as through a speaker, e.g., in a handset of the mobile computing device 250. Such sound may include sound from voice telephone calls, may include recorded sound (e.g., voice messages, music files, etc.) and may also include sound generated by applications operating on the mobile computing device 250.

The mobile computing device 250 may be implemented in a number of different forms, as shown in the figure. For example, it may be implemented as a cellular telephone 280. It may also be implemented as part of a smart-phone 282, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In certain embodiments, the system comprises a physical biorepository 290 (comprising one or more cell storage containers) in communication with any of the computer system arrangements of FIG. 1 or 2.

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for the convenience of the reader and are not intended to be limiting with respect to the claimed subject matter.

Documents are incorporated herein by reference as noted. Where there is any discrepancy in the meaning of a particular term, the meaning provided in the Definition section above is controlling.

Certain embodiments of the present invention are described herein. It is, however, expressly noted that the present invention is not limited to these embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description. Therefore, the disclosure should not be limited to certain implementations, but rather should be limited only by the spirit and scope of the following claims.

### Equivalents

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An ex vivo method for detection of a genetic disease or condition in a subject and/or determination of one or more treatment options for the subject, the method comprising:
accessing genotyping data for the subject;
obtaining a manufactured sample of induced pluripotent stem cells (iPSCs) derived from a biological sample provided by the subject;
creating a plurality of testing samples, wherein each of the plurality of testing samples comprises ex vivo differentiated cells derived from the manufactured sample;
performing a functional assay to test efficacies of one or more substances using the plurality of testing samples; and
determining an existence or absence of the genetic disease or condition in the subject and/or determining one or more treatment options for the subject having the genetic disease or condition based at least in part on the functional assay and based at least in part, directly or indirectly, on the genotyping data for the subject.

2. The method of claim 1, comprising generating genotyping data from measurements of genetic material derived from the manufactured sample of iPSCs.

3. The method of claim 1 or 2, wherein the step of performing the functional assay to test efficacies comprises using the plurality of testing samples to test one or more individual substances or combination of substances, wherein each of the plurality of testing samples receives a distinct substance or a distinct combination of substances.

4. The method of any one of claims 1 to 3, comprising determining the one or more substances for use in performing the functional assay based at least in part on the genotyping data.

5. The method of any one of claims 1 to 4, comprising generating genotyping data from measurements of variants of the subject of one or more SNPs associated with the genetic disease.

6. The method of any one of claims 1 to 5, further comprising the step of:
generating the sample of induced pluripotent stem cells from the biological sample provided by the subject.

7. The method of any one of claims 1 to 6, wherein the ex vivo differentiated cells are cardiomyocytes, wherein the ex vivo differentiated cells of each testing sample of the plurality of testing samples have a synchronous heartbeat.

8. The method of claim 7, wherein the genetic disease or condition is Long QT syndrome.

9. The method of any one of claims 1 to 6, wherein the genetic disease or condition is a member selected from the group consisting of: Charcot-Marie-Tooth disorder, 47 XYY syndrome, Jacobs syndrome, Brugada syndrome, Turner syndrome, Fragile-X syndrome, Neurofibromatosis-1 muscular dystrophy (Duchenne and Becker type), Hereditary sensory and autonomic neuropathy 3, chromosome 22q11.2 deletion syndrome, alpha 1-antitrypsin deficiency, Long QT, and hereditary hemorrhagic telangiectasia.

10. The method of any one of claims 1 to 9, comprising repeating the step of performing the functional assay.

11. The method of any one of claims 1 to 10, comprising repeating the step of accessing the genotyping data.

12. A method for creating a database of treatment options based on genotypes for use in treatment of subjects with genetic diseases or conditions, the method comprising:
accessing genotyping data for a plurality of subjects;
obtaining, for each subject in the plurality of subjects, a manufactured sample of induced pluripotent stem cells (iPSCs) derived from a biological sample provided by the subject;
deriving ex vivo differentiated cells from each manufactured sample;
performing a plurality of functional assays with the ex vivo differentiated cells and one or more substances;
determining one or more treatment options for each of a plurality of genotypes based on the plurality of functional assays; and
generating a mapping by compiling determinations of the one or more treatment options and corresponding genotypes of the plurality of genotypes.

13. The method of claim 12, wherein the biological sample of the subject comprises a member selected from the group consisting of: a cheek swab, a blood sample, a urine sample, and a tissue sample of the subject.

14. The method of claim 12 or 13, wherein the step of performing the plurality of functional assays comprises testing one or more individual substances or combination of substances, wherein each of the plurality of functional assays receives a distinct substance or a distinct combination of substances.

15. The method of any one of claims 12 to 14, wherein the step of generating the mapping comprises creating a database.

16. The method of any one of claims 12 to 15, comprising:
performing, repeatedly, the plurality of functional assays for each subject with the ex vivo differentiated cells of the subject, wherein performing the plurality of functional assays comprises:
selecting the one or more substances for each subject based on the genotyping data for the subject.

17. A method for creating a database of long-term treatment options based on genotypes for use in long-term treatment of subjects with genetic diseases or conditions, the method comprising:
accessing genotyping data for a plurality of subjects;
obtaining, for each subject in the plurality of subjects, a manufactured sample of induced pluripotent stem cells (iPSCs) derived from a biological sample provided by the subject;
creating, for each subject in the plurality of subjects, a plurality of testing samples, wherein each of the plurality of testing samples comprises ex vivo differentiated cells derived from the manufactured sample for the subject;
performing a functional assay to test efficacies of one or more substances for long-term treatment using the plurality of testing samples for each subject of the plurality of subjects, wherein each of the plurality of testing samples receives a distinct substance or a distinct combination of substances, wherein the plurality of substances is selected based on the genotyping data for the subject;
determining one or more individual long-term treatment options to recommend to treat the genetic disease or condition that each subject of the plurality of subjects has based, at least in part, on the functional assay for the subject;
determining one or more preferred general long-term treatment options for each of a plurality of genotypes based on the genotyping data for each subject of the plurality of subjects and the one or more individual long-term treatment options; and
generating a mapping by compiling determinations of the one or more preferred general long-term treatment options and corresponding genotypes of the plurality of genotypes.

18. The method of claim 17, wherein the step of performing the functional assay to test efficacies comprises using the plurality of testing samples to test one or more individual substances or combination of substances.

19. The method of claim 17 or 18, wherein the step of generating the mapping comprises creating a database.

20. The method of any one of claims 17 to 19, comprising the step of:
generating the genotyping data for each subject of the plurality of subjects using the manufactured sample for each subject.

21. The method of claim 20, wherein the step of generating the genotyping data for each subject in the plurality of subjects comprises determining at least a portion of a genotype corresponding to a genetic disease or condition for each subject.

22. The method of any one of claim 20 or 21, comprising generating genotyping data from measurements of variants of one or more SNPs associated with the genetic disease or condition of the subject.

23. The method of any one of claims 17 to 22, comprising determining the one or more substances for use in performing the functional assay for each subject in the plurality of subjects based at least in part on the genotyping data for the subject.

24. The method of any one of claims 12 to 23, comprising repeatedly monitoring the genotyping data of each subject in the plurality of subjects.

25. A method of long term storage of a plurality of reserves of immortalized cell lines extracted and/or generated from biological samples of a plurality of individuals, the method comprising:
storing, by a processor of a computer device, genotyping data corresponding to each of the plurality of reserves of immortalized cell lines corresponding to the plurality of individuals, said plurality of reserves contained in a bank.

26. The method of claim 25, further comprising storing the plurality of reserves of immortalized cell lines in the bank.

27. The method of claim 25 or 26, wherein the plurality of reserves of immortalized cell lines comprises induced pluripotent stem cells (iPSCs) and/or undifferentiated cells.

28. The method of any one of claims 25-27, wherein the biological samples comprises saliva, blood, tissue, cheek cells, urine, and/or hair samples.

29. The method of claim 25, comprising, triggering, by the processor, a notification.

30. The method of claim 29, wherein the triggering of the notification occurs at one or more predetermined intervals of time, or upon occurrence of an event.

31. The method of claim 30, wherein the triggering of the notification comprises issuing an alert associated with a reserve of the plurality of reserves of immortalized cell lines.

32. The method of claim 31, wherein the alert is an alert of low reserve amount comprising an identification of the reserve, an individual associated with the reserve, and/or a meter value associated with a low reserve amount associated with the individual.

33. The method of claim 31 or 32, wherein the issuing of the alert associated with a reserve of the plurality of reserves of immortalized cell lines comprises an alert of new genetic tests, advancements in treatments relevant to an individual associated with the reserve, and/or access genetic material without requiring individual to provide another sample.

34. A repository of characterized immortalized cell lines that are capable of being cultured, expanded, stored, differentiated, and queried over a long period of time for repeated genetic analysis, tissue creation, determination of treatment options, and/or lab-on-a-chip applications.

35. The repository of claim 34, wherein the immortalized cell lines comprise undifferentiated cells and/or iPSCs.

36. The repository of claim 34 or 35, wherein the immortalized cell lines are stored at a temperature of about −195° C.

37. The repository of any one of claims 34 to 36, wherein the immortalized cell lines are stored in a liquid nitrogen storage tank and/or a freezer.

38. A method for using a repository of characterized immortalized cell lines that are capable of being cultured, expanded, stored, differentiated, and queried over a long period of time for repeated genetic analysis, tissue creation, determination of treatment options, and/or lab-on-a-chip applications, the method comprising:
for each of a plurality of individuals whose immortalized cell lines are contained in the repository, accessing, by a processor of a computer device, genotyping data corresponding to each of the immortalized cell lines stored in a reserve of the repository for use in in vitro and/or in vivo clinical procedures.

39. The use of the repository of claim 38, wherein the clinical procedures comprise gene therapy, cell or tissue transplant, mesenchymal stem cell transplant, bone marrow transplant, and cosmetic surgery.

40. A method for using a repository of characterized immortalized cell lines that are capable of being cultured, expanded, stored, differentiated, and queried over a long period of time for repeated genetic analysis, tissue creation, determination of treatment options, and/or lab-on-a-chip applications, the method comprising:
for each of a plurality of individuals whose immortalized cell lines are contained in the repository, accessing, by a processor of a computer device, genotyping data corresponding to each of the immortalized cell lines stored in a reserve of the repository for use in in vitro and/or in vivo pre-clinical studies.

41. A system for detection of a genetic disease or condition in a subject and/or determination of one or more treatment options for the subject, the system comprising:
a processor; and
a memory having instructions stored thereon, wherein the instructions, when executed by the processor, cause the processor to:
access genotyping data for the subject; and
determine an existence or absence of the genetic disease or condition in the subject and/or determine one or more treatment options for the subject having the genetic disease or condition, (i) based at least in part on a functional assay performed on a plurality of testing samples comprising ex vivo differentiated cells derived from a manufactured sample of induced pluripotent stem cells (iPSCs) derived from a biological sample provided by the subject, and (ii) based at least in part, directly or indirectly, on the genotyping data for the subject.

42. The system of claim 41, wherein the instructions, when executed by the processor cause the processor to generate genotyping data from measurements of genetic material derived from the manufactured sample of iPSCs.

43. The system of claim 41 or 42, wherein the functional assay is or has been performed to test efficacies of one or more individual substances or combination of substances using the plurality of testing samples, each of the plurality of testing samples receiving a distinct substance or a distinct combination of substances.

44. The system of any one of claims 41 to 43, wherein the instructions, when executed by the processor cause the processor to determine the one or more substances for use in performing the functional assay based at least in part on the genotyping data.

45. The system of any one of claims 41 to 44, wherein the instructions, when executed by the processor cause the processor to generate genotyping data from measurements of variants of the subject of one or more SNPs associated with the genetic disease.

46. The system of any one of claims 41 to 45, wherein the ex vivo differentiated cells are cardiomyocytes, wherein the ex vivo differentiated cells of each testing sample of the plurality of testing samples have a synchronous heartbeat.

47. The system of claim 46, wherein the genetic disease or condition is Long QT syndrome.

48. The system of any one of claims 41 to 45, wherein the genetic disease or condition is a member selected from the group consisting of: Charcot-Marie-Tooth disorder, 47 XYY syndrome, Jacobs syndrome, Brugada syndrome, Turner syndrome, Fragile-X syndrome, Neurofibromatosis-1 muscular dystrophy (Duchenne and Becker type), Hereditary sensory and autonomic neuropathy 3, chromosome 22q11.2 deletion syndrome, alpha 1-antitrypsin deficiency, Long QT, and hereditary hemorrhagic telangiectasia.

49. The system of any one of claims 41 to 48, wherein the functional assay is or has been performed repeatedly.

50. The system of any one of claims 41 to 49, wherein the instructions, when executed by the processor, cause the processor to repeatedly access the genotyping data for the subject.

51. A system for creating a database of treatment options based on genotypes for use in treatment of subjects with genetic diseases or conditions, the system comprising:
a processor; and
a memory having instructions stored thereon, wherein the instructions, when executed by the processor, cause the processor to:
access genotyping data for a plurality of subjects;
determine one or more treatment options for each of a plurality of genotypes (i) based at least in part on a plurality of functional assays performed on ex vivo differentiated cells derived from a manufactured sample of induced pluripotent stem cells (iPSCs) derived from a biological sample provided by each subject in a plurality of subjects, and (ii) based at least in part, directly or indirectly, on the genotyping data for each of the plurality of subjects; and
generate a mapping by compiling determinations of the one or more treatment options and corresponding genotypes of the plurality of genotypes.

52. The system of claim 51, wherein the biological sample is a cheek swab, a blood sample, a urine sample, and/or a tissue sample.

53. The system of claim 51 or 52, wherein the plurality of functional assays are or have been performed to test efficacies of one or more individual substances or combination of substances using the differentiated cells, each of the plurality of functional assays receiving a distinct substance or a distinct combination of substances.

54. The system of any one of claims 51 to 53, wherein the instructions, when executed by the processor cause the processor to generate a database of mappings of determinations of the one or more treatment options and corresponding genotypes of the plurality of genotypes.

55. The system of any one of claims 51 to 54, wherein the instructions, when executed by the processor cause the processor to:
select the one or more substances for each subject in a plurality of subjects based on the genotyping data for the subject to perform, repeatedly, the plurality of functional assays for each subject with the ex vivo differentiated cells of the subject.

56. A system for creating a database of long-term treatment options based on genotypes for use in long-term treatment of subjects with genetic diseases or conditions, the system comprising:
a processor; and
a memory having instructions stored thereon, wherein the instructions, when executed by the processor, cause the processor to:
access genotyping data for a plurality of subjects;
determine one or more individual long-term treatment options to recommend to treat the genetic disease or condition that each subject of the plurality of subjects has (i) based at least in part on a functional assay to test long-term treatment efficacies of one or more substances performed on a plurality of testing samples for each subject of the plurality of subjects, wherein the plurality of testing samples comprising ex vivo differentiated cells derived from a manufactured sample of induced pluripotent stem cells (iPSCs) derived from a biological sample provided by each subject in the plurality of subjects, and (ii) based at least in part, directly or indirectly, on the genotyping data for the subject;
determine one or more preferred general long-term treatment options for each of a plurality of genotypes based on the genotyping data for each subject of the plurality of subjects and the one or more individual long-term treatment options; and
generate a mapping by compiling determinations of the one or more preferred general long-term treatment options and corresponding genotypes of the plurality of genotypes.

57. The system of claim 56, wherein the functional assay is or has been performed to test efficacies of one or more individual substances or combination of substances using the plurality of testing samples, each of the plurality of testing samples receiving a distinct substance or a distinct combination of substances.

58. The system of claim 56 or 57, wherein the instructions, when executed by the processor cause the processor to generate a database of mappings of determinations of the one or more preferred general long-term treatment options and corresponding genotypes of the plurality of genotypes.

59. The system of any one of claims 56 to 58, wherein the instructions, when executed by the processor cause the processor to generate genotyping data for each subject of the plurality of subjects using the manufactured sample for each subject.

60. The system of claim 59, wherein the instructions, when executed by the processor cause the processor to generate genotyping data for each subject in the plurality of subjects from measurements of at least a portion of the subject's genetic material corresponding to a genetic disease or condition.

61. The system of claim 59 or 60, wherein the instructions, when executed by the processor cause the processor to generate genotyping data from measurements of variants of one or more SNPs associated with the genetic disease or condition of the subject.

62. The system of any one of claims 56 to 61, wherein the instructions, when executed by the processor cause the processor to determine the one or more substances for use in the functional assay for each subject in the plurality of subjects based at least in part on the genotyping data for the subject.

63. The system of any one of claims 51 to 62, wherein the instructions, when executed by the processor cause the processor to repeatedly monitor the genotyping data of each subject in the plurality of subjects.

64. A system of long-term storage of a plurality of reserves of immortalized cell lines extracted and/or generated from biological samples of a plurality of individuals, the system comprising:
a processor; and
a memory having instructions stored thereon, wherein the instructions, when executed by the processor, cause the processor to store genotyping data corresponding to each of the plurality of reserves of immortalized cell lines contained in a bank corresponding to the plurality of individuals.

65. The system of claim 64 further comprising long-term storage of the plurality of reserves of immortalized cell lines in the bank.

66. The system of claim 64 or 65, wherein the plurality of reserves of immortalized cell lines comprise induced pluripotent stem cells (iPSCs) and/or undifferentiated cells.

67. The system of any one of claims 64 to 66, wherein the plurality of reserves of immortalized cell lines comprise immortalized cell lines derived from saliva, blood, tissue, cheek cells, urine, and/or hair samples of the plurality of individuals.

68. The system of claim 64, wherein the instructions, when executed by the processor, cause the processor to trigger a notification.

69. The system of claim 68, wherein the instructions, when executed by the processor cause the processor to trigger the notification at one or more predetermined intervals of time, or upon occurrence of an event.

70. The system of claim 68 or 69, wherein the notification comprises issuing an alert associated with a reserve of the plurality of reserves of immortalized cell lines.

71. The system of claim 70, wherein the instructions, when executed by the processor cause the processor to issue the alert, wherein the alert is an alert of low reserve amount, wherein the alert of low reserve amount comprises an identification of the reserve, an individual associated with the reserve, and/or a meter value associated with a low reserve amount associated with the individual.

72. The system of claim 70 or 71, wherein the instructions, when executed by the processor cause the processor to issue the alert, wherein the alert is an alert of new genetic tests, advancements in treatments relevant to an individual associated with the reserve, and/or access genetic material without requiring individual to provide another sample.

73. A system for using a repository of characterized immortalized cell lines that are capable of being cultured, expanded, stored, differentiated, and queried over a long period of time for repeated genetic analysis, tissue creation, determination of treatment options, and/or lab-on-a-chip applications, the system comprising:
a processor of a computing device; and
a memory comprising instructions stored thereon, wherein the instructions, when executed by the processor, cause the processor to:
access, for each of a plurality of individuals whose immortalized cell lines are contained in the repository, genotyping data corresponding to each of the immortalized cell lines stored in a reserve of the repository for use in in vitro and/or in vivo clinical procedures.

74. The system of claim 73, wherein the clinical procedures comprise gene therapy, cell or tissue transplant, mesenchymal stem cell transplant, bone marrow transplant, and cosmetic surgery.

75. A system for using a repository of characterized immortalized cell lines that are capable of being cultured, expanded, stored, differentiated, and queried over a long period of time for repeated genetic analysis, tissue creation, determination of treatment options, and/or lab-on-a-chip applications, the system comprising:
a processor of a computing device; and
a memory comprising instructions stored thereon, wherein the instructions, when executed by the processor, cause the processor to:
access, for each of a plurality of individuals whose immortalized cell lines are contained in the repository, genotyping data corresponding to each of the immortalized cell lines stored in a reserve of the repository for use in in vitro and/or in vivo pre-clinical studies.
